(19) United States
(12) Patent Application Publication
(10) Pub. No.: US 2023/0138844 A1
(43) Pub. Date: May 4, 2023

(54) SYSTEMS FOR SUPPRESSION OF BALLISTOCARDIOGRAM ARTIFACTS IN ELECTROENCEPHALOGRAPHY SIGNALS VIA DYNAMIC HEARTBEAT MODELING

(71) Applicant: SUNNYBROOK RESEARCH INSTITUTE, Toronto (CA)

(72) Inventor: FA-HSUAN LIN, TORONTO (CA)

(21) Appl. No.: 17/700,694

(22) Filed: Mar. 22, 2022

(30) Foreign Application Priority Data
Nov. 2, 2021 (DE) 20 2021 105 989.3

(51) Int. Cl. A61B 5/00; A61B 5/369; A61B 5/291; A61B 5/346

(52) U.S. Cl. CPC A61B 5/7214; A61B 5/291; A61B 5/346; A61B 5/369

(57) ABSTRACT

Systems are provided that employ dynamic modeling of heartbeats to process electroencephalography (EEG) signals for the suppression of BCG artifacts. The system may be configured to generate an instantaneous EEG correction for ballistocardiogram (BCG) artifact subtraction, the correction being modeled for a selected latency within a selected cardiac cycle. Cardiac cycles with similar EKG signals at the selected latency to that of the selected cardiac cycle are identified and the EEG signals from these similar cardiac cycles, at the selected latency, are employed to generate a modeled EEG signal that represents the instantaneous contribution from the BCG artifact. Accordingly, the system models BCG artifacts by pooling EEG signals at time instants with similar cardiac dynamics. The resulting modeled EEG signal is taken as the estimated BCG artifact and subtracted from the measured EEG signals to generate artifact-suppressed EEG signals.

### CROSS-REFERENCE TO RELATED APPLICATIONS

This application claims priority to German Patent Application No. 20 2021 105 989.3, titled “SYSTEMS FOR SUPPRESSION OF BALLISTOCARDIOGRAM ARTIFACTS IN ELECTROENCEPHALOGRAPHY SIGNALS VIA DYNAMIC HEARTBEAT MODELING” and filed on Nov. 2, 2021, the entire contents of which is incorporated herein by reference.

### BACKGROUND

The present disclosure relates to the processing of electroencephalography signals. More particularly, the present disclosure relates to systems and methods for the suppression of ballistocardiogram artifacts in electroencephalography data acquired during magnetic resonance imaging.

Electroencephalography (EEG) recording and functional magnetic resonance imaging (fMRI) in the same setting allows for the characterization of neuronal and hemodynamic signals. This technique has been fruitful in elucidating neurovascular coupling effects and localizing epileptiform activity generators. However, EEG recorded concurrently with magnetic resonance imaging (MRI) is seriously deteriorated by the induced voltage caused by the switching of MRI gradient coils. Gradient artifacts in EEG data can be effectively suppressed by artifact template estimation and subtraction or by minimizing the duty cycles of MRI acquisition.

Ballistocardiogram (BCG) artifacts also occur when EEG data are collected inside an MRI system. In the present context, the BCG artifact is the EEG signal fluctuation that is obtained within a strong static magnetic field, when scalp electrodes move due to cardiac pulsatility. The amplitude of the BCG artifact ranges between 150 and 200 µV, which is larger than the typical electrophysiological activity of interest (approximately 1-10 µV). The duration of the BCG artifact typically lasts for approximately 1 second. Due to its high amplitude, significant spectrotemporal overlap with neurophysiologic signals, and large variations in the waveform shape, timing, and intensity across time and electrode locations, BCG artifacts substantially reduce the sensitivity and specificity for detecting the EEG signals of interest. The amplitude of the artifact scales with the field strength of MRI. Therefore, suppressing BCG artifacts becomes more pressing when EEG data are recorded inside a high-field (≥ 3 T) MRI system.

Among methods to suppress BCG artifacts, several estimate an artifact “template” waveform and then subtract the template from the contaminated EEG signals. The estimation of an artifact template is based on the collection of EEG signals temporally aligned to a specific time point in the cardiac cycle, such as the peak of the QRS complex. Either the average or a combination of aligned EEG signal segments are taken as the artifact template. Although widely used, these methods do not consider the variations in QRS complex timing, EKG shape, and EKG amplitudes, which may lead to out-of-phase artifact subtraction, systematic errors, and large residuals.

Methods based on Principal Component Analysis (PCA) and Independent Component Analysis (ICA) provide alternative ways to suppress BCG artifacts. However, these methods require a sufficient number of sensors to allow for the decomposition of the measurements into “signal” and “noise” components. Categorizing the decomposed components as either “signal” or “noise” typically requires a heuristic definition, which may reduce the performance of BCG artifact suppression. More recently, a reference-free harmonic regression modeling approach was proposed to suppress BCG artifacts. This method shows comparable performance with the Optimal Basis Set (OBS) method in revealing evoked potentials, while suppressing BCG artifacts (21% in harmonic regression vs. 26% in OBS).

### SUMMARY

Systems are provided that employ dynamic modeling of heartbeats to process electroencephalography (EEG) signals for the suppression of BCG artifacts. The system may be configured to generate an instantaneous EEG correction for ballistocardiogram (BCG) artifact subtraction, the correction being modeled for a selected latency within a selected cardiac cycle. Cardiac cycles with similar EKG signals at the selected latency to that of the selected cardiac cycle are identified and the EEG signals from these similar cardiac cycles, at the selected latency, are employed to generate a modeled EEG signal that represents the instantaneous contribution from the BCG artifact. Accordingly, the system models BCG artifacts by pooling EEG signals at time instants with similar cardiac dynamics. The resulting modeled EEG signal is taken as the estimated BCG artifact and subtracted from the measured EEG signals to generate artifact-suppressed EEG signals.

Accordingly, in a first aspect, there is provided a system for performing suppression of ballistocardiogram artifacts in electroencephalography data (EEG data), the system comprising:
processing circuitry comprising at least one processor and associated memory, the memory comprising instructions executable by the at least one processor for performing operations comprising:
   receiving the EEG data and corresponding electrocardiogram data (EKG data);
   processing the EKG data to identify a plurality of cardiac cycles, each cardiac cycle having an associated EKG waveform segment;
   generating a modeled value of the EEG data for a selected latency value within a selected cardiac cycle having an associated EKG waveform segment, the modeled value being associated with the ballistocardiogram artifact contribution to the EEG data, by:
      processing EKG waveform segments of cardiac cycles other than the selected cardiac cycle to identify one or more similar cardiac cycles that have similar EKG waveform segment values, proximal to the selected latency value, to those of selected cardiac cycle;
      employing values of the EEG waveforms of the similar cardiac cycles at the selected latency value to generate a modeled value of the EEG waveform at the selected latency value for the selected cardiac cycle; and
   subtracting the modeled value of the EEG waveform from the EEG data at the selected latency value within the selected cardiac cycle to obtain corrected EEG data having BCG artifact suppressed at the selected latency value.

In some example implementations of the system, the processor is configured such that processing EKG waveform segments of cardiac cycles other than the selected cardiac cycle to identify one or more similar cardiac cycles that have similar EKG waveform segment values, proximal to the selected latency value, to those of selected cardiac cycle, comprises:
obtaining, for each cardiac cycle, a set of EKG values corresponding to a set of latency values proximal to the selected latency value;
processing the sets of EKG values of cardiac cycles other than the selected cardiac cycle to identify one or more similar cardiac cycles that have a set of EKG values similar to those of selected cardiac cycle.

In some example implementations of the system, the processor is configured such that the one or more similar cardiac cycles are identified according to a distance-based similarity measure associated with a multi-dimensional representation of each set of EKG values, with each EKG value of a given set of EKG values being associated with a different dimension. The processor may be configured such that the distance-based similarity measure is computed according to Euclidean distance.

In some example implementations of the system, the processor is configured such that contributions of the EEG waveform values corresponding to the similar cardiac cycles are weighted according to similarity of the EKG waveform values to those of the selected cardiac cycle at the selected latency value to generate modeled value of the EEG waveform.

In some example implementations of the system, the processor is configured such that the latency is determined relative to a QRS complex associated with each cardiac cycle.

In some example implementations of the system, the processor is further configured to perform BCG artifact suppression at a plurality of latency values within the selected cardiac cycle.

In some example implementations of the system, the processor is further configured to perform BCG artifact suppression at a plurality of latency values within a plurality of selected cardiac cycles.

In some example implementations, the system further comprises an EKG sensor and an EEG electrode array, wherein the processing circuitry is operatively coupled to the EKG sensor and the EEG electrode array. The system may further comprise a magnetic resonance imaging scanner, wherein the processing circuitry is operatively coupled to the magnetic resonance imaging scanner.

In another aspect, there is provided a system for performing suppression of ballistocardiogram artifacts in electroencephalography data (EEG data), the system comprising:
processing circuitry comprising at least one processor and associated memory, the memory comprising instructions executable by the at least one processor for performing operations comprising:
   receiving the EEG data and corresponding electrocardiogram data (EKG data);
   processing the EKG data to identify a plurality of cardiac cycles, each cardiac cycle having an associated EKG waveform segment;
   employing a temporal mapping to map the EKG data to a mapped EKG dataset such that each EKG waveform segment of the mapped EKG dataset is characterized by a respective cardiac cycle index and a latency parameter denoting latency within the respective cardiac cycle;
   processing the mapped EKG dataset to generate, for each cardiac cycle, a corresponding multi-dimensional manifold, each dimension of the manifold being associated with a respective mapped EKG dataset having a unique time lag;
   processing the manifolds to identify one or more similar cardiac cycles that have similar values, at a selected latency value, to that of a selected cardiac cycle at the selected latency value;
   employing values of the EEG waveforms of the similar cardiac cycles at the selected latency value to generate a modeled value of the EEG waveform at the selected latency value for the selected cardiac cycle; and
   subtracting the modeled value of the EEG waveform from the EEG data at the selected latency value within the selected cardiac cycle to obtain corrected EEG data having BCG artifact suppressed at the selected latency value.

A further understanding of the functional and advantageous aspects of the disclosure can be realized by reference to the following detailed description and drawings.

### BRIEF DESCRIPTION OF THE DRAWINGS

Embodiments will now be described, by way of example only, with reference to the drawings, in which:

FIG. 1 is a flow chart illustrating an example processing method of performing suppression of ballistocardiogram artifacts in EEG data.

FIGS. 2A, 2B, 2C, 2D and 2E illustrate the procedure of building a two-dimensional manifold K_{EKG} (e = 2) and seeking five nearest neighbors (n = 5) to derive ŝ_{EEG}(t).

FIG. 3 is a block diagram illustrating an example system for performing suppression of ballistocardiogram artifacts in EEG data during magnetic resonance imaging.

FIG. 4 plots the difference of the evoked response 15-Hz oscillatory power between DMH and OBS processing for different DMH parameter combinations. Differences were averaged across O1, O2, and Oz electrodes and participants. Positive differences correspond to larger 15-Hz oscillatory power by DMH compared to OBS.

FIGS. 5A and 5B show average evoked responses (FIG. 5A) and average 15-Hz time-frequency representation amplitude (FIG. 5B) across O1, O2, and Oz electrodes.

FIG. 6 shows spatial distributions of 15-Hz time-frequency representation of the estimated neuronal current power over cortical surfaces. Distributions were Z scores averaged over the time interval from +200 ms to +1000 ms after the visual stimulus onset, with respect to the baseline pre-stimulus interval.

FIG. 7 plots time courses of the estimated neuronal current over cortical surfaces at the visual cortex with DMH or OBS to suppress BCG artifacts. The boundary of the visual cortex is illustrated by pink cortical patches in the inlet of the top panel.

FIGS. 8A and 8B show time-frequency representations of the estimated neuronal current at the visual cortex using DMH or OBS to suppress BCG artifacts. (FIG. 8A): Plots of time-frequency representations between 5 Hz and 30 Hz. (FIG. 8B): Plots of noise-normalized time-frequency representations between 5 Hz and 30 Hz.

FIG. 9 shows receiver-operating characteristic curves of the 15-Hz time-frequency representation averaged between 200 ms and 1000 ms after the visual stimulus onset. SSVEPs (steady-state visual evoked response paradigm) with DMH have larger areas under the ROC curves in EPI-EEG (concurrent recording of EEG with echo-planar imaging), SMS-Inl-EEG (concurrent recording of EEG with fast MRI acquisition using simultaneous multi-slice and inverse imaging methods), and Inside-MRI (inside the MRI system without imaging) conditions than OBS.

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms “comprises” and “comprising” are to be construed as being inclusive and open ended, and not exclusive. Specifically, when used in the specification and claims, the terms “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately” are meant to cover variations that may exist in the upper and lower limits of the ranges of values, such as variations in properties, parameters, and dimensions. Unless otherwise specified, the terms “about” and “approximately” mean plus or minus 25 percent or less.

It is to be understood that unless otherwise specified, any specified range or group is as a shorthand way of referring to each and every member of a range or group individually, as well as each and every possible sub-range or sub-group encompassed therein and similarly with respect to any sub-ranges or sub-groups therein. Unless otherwise specified, the present disclosure relates to and explicitly incorporates each and every specific member and combination of sub-ranges or sub-groups.

As used herein, the term “on the order of”, when used in conjunction with a quantity or parameter, refers to a range spanning approximately one tenth to ten times the stated quantity or parameter.

As noted above, the ballistocardiogram (BCG), defined as the induced electric potentials by the head motion originating from heartbeats, is a prominent source of noise in electroencephalography (EEG) data during magnetic resonance imaging (MRI). The BCG artifact scales with MRI field strength and limits the sensitivity of detecting neurophysiological signatures from the EEG concurrently recorded with MRI. Although methods have been proposed to suppress the BCG artifact, the variability of cardiac cycles and head motion across time and subjects can impair the ability of such methods to provide a robust correction.

The present disclosure provides systems and associated methods that employ the dynamic modeling of heartbeats, henceforth referred to as the DMH system/method, to suppress BCG artifacts in EEG signals, with a particular benefit for denoising EEG signals recorded during simultaneous magnetic resonance image acquisition. According to various example embodiments disclosed below, an instantaneous EEG signal correction for BCG artifact subtraction is modeled for a specific latency (phase) within a cardiac cycle by combining EEG signals at the same latency (phase) within the other cardiac cycles that exhibit similar dynamic features. The present example DMH method thus models BCG artifacts by pooling EEG signals at time instants with similar cardiac dynamics. The resulting modeled EEG signal is taken as the estimated BCG artifact and subtracted from the original recording to generate artifact-suppressed EEG signals.

The present example embodiments provide a clear improvement over known BCG suppression methods that are capable, for example, of improving the detection of event-related potentials (ERPs) collected inside a high-field (3T) MRI scanner. ERPs have been extensively used to characterize brain cognitive functions and dysfunctions by cognitive neuroscientists, psychiatrists, and neurologist. As demonstrated below, the improvement of the steady-state visual evoked potentials by DMH is about 130% of that achieved by the OBS method.

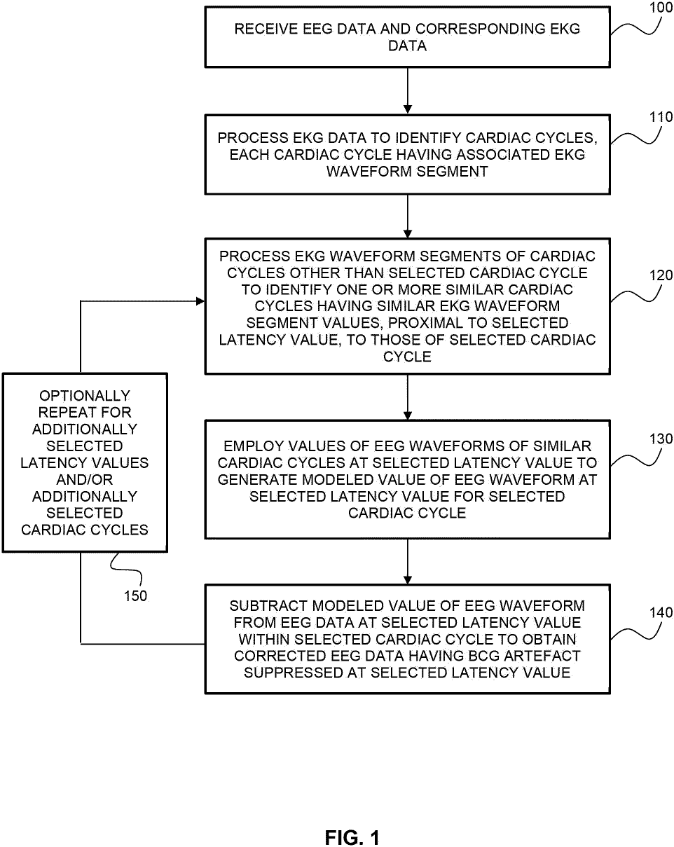

Referring now to FIG. 1, an example flow chart is provided that illustrates an example method of performing BCG suppression of EEG signals via dynamic heart modeling. In step 100, the EEG data and corresponding EKG data are received. As noted above, the EEG data and corresponding EKG data may be recorded concurrently while performing magnetic resonance imaging, such that the EEG data includes BCG artifacts.

In step 110, the EKG is processed to identify a plurality of cardiac cycles, with each cardiac cycle having an associated EKG waveform segment. Example methods of identifying cardiac cycles in EKG data are described in further detail below. Identifying the cardiac cycles within the EKG data enables the EKG data to be represented as a set of EKG waveform segments, with each EKG waveform segment being associated with a respective cardiac cycle and characterized by a latency parameter (denoting latency within the respective cardiac cycle).

The EKG and EEG data may then be employed to determine, for a selected latency within a selected cardiac cycle, a modeled EEG waveform value that represents the BCG artifact. This modeled EEG waveform value may be subsequently subtracted from the measured EEG waveform value, for the selected latency within the selected cardiac cycle, to obtain a corrected EEG waveform value, at the selected latency within the selected cardiac cycle, for which the BCG artifact has been suppressed.

The modeled EEG waveform value that represents the BCG artifact at the selected latency for the selected cardiac cycle may be determined by performing steps 120 and 130 in FIG. 1. In step 120, the EKG waveform segments corresponding to cardiac cycles other than the selected cardiac cycle are processed to identify cardiac cycles that have similar EKG waveform values, for latency values proximal to the selected latency value, to EKG waveform values of the selected cardiac cycle (for latency values proximal to the selected latency value). This step thus identifies cardiac cycles that are similar at the selected latency value. In step 130, the values of the EEG waveform segments for one or more of the similar cardiac cycles, at the selected latency value, are employed to generate the modeled value of the EEG waveform at the selected latency value for the selected cardiac cycle.

In some example implementations, the similar cardiac cycles may be identified by obtaining, for each cardiac cycle, a set of EKG waveform values corresponding to a set of latency values proximal to the selected latency value, and processing the sets of EKG values corresponding to the cardiac cycles other than the selected cardiac cycle to identify one or more similar cardiac cycles that have a respective set of EKG values that are similar to the set of EKG values of the selected cardiac cycle proximal to the selected latency value.

For example, the one or more similar cardiac cycles may be identified according to a distance-based similarity measure associated with a multi-dimensional representation of each set of EKG values, with each EKG value of a given set of EKG values being associated with a different dimension. An example of such an implementation is described in detail below for the example case of two dimensions. In some example implementations, the distance measure may be a Euclidean distance measure.

Many different implementations can be employed to process the values of the EEG waveform segments for one or more of the similar cardiac cycles, at the selected latency value, to generate the modeled value of the EEG waveform at the selected latency value for the selected cardiac cycle. For example, one or more EEG waveform values that correspond to the cardiac cycles with the highest similarity may be employed to generate the modeled value of the EEG waveform. In some example implementations, the contributions of the EEG waveform values corresponding to a plurality of similar cardiac cycles may be weighted, according to a similar measure associated with a similarity of the EKG waveform values, such as a distance-based similarity measure.

Having generated the modeled EEG waveform value that represents the BCG artifact at the selected latency for the selected cardiac cycle, the modeled EEG waveform value may be subtracted from the measured EEG waveform value, for the selected latency value within the selected cardiac cycle, to obtain a corrected EEG waveform value, at the selected latency value within the selected cardiac cycle, for which the BCG artifact has been suppressed, as per step 140.

As shown at step 150, the processing implemented in steps 120 to 140 may be repeated one or more times to generate additional modeled EEG waveform values that represent the BCG artifact to the EEG waveform at other latency values within the selected cardiac cycle, and these additional modeled EEG waveform values may be subtracted to generate BCG-artifact-suppressed EEG values at these other latency values, and this process may be repeated for latency values within one or more additional selected cardiac cycles.

In some example implementations, the cardiac cycles are identified according to peaks of the QRS complex within the EKG data. For example, as described in Pan et al. (J. Pan and W. J. Tompkins, “A Real-Time QRS Detection Algorithm,” in IEEE Transactions on Biomedical Engineering, vol. BME-32, no. 3, pp. 230-236, March 1985, doi: 10.1109/TBME.1985.325532), the Pan-Tompkins algorithm allows for the real-time detection of the cardiac QRS complexes based on the slope, width, and amplitude of the EKG waveform via digital band-pass filtering.

The collected EEG and EKG waveforms may be synchronized, for example by digitizing both sets of waveforms with a common clock. In some example implementations, both EEG and EKG waveforms are fed to the same system allowing for parallel analog-to-digital data conversion with the sampling rate up to 1000 Hz.

An example implementation of the method shown in FIG. 1 is described in detail below using an example and illustrative mathematical framework. It will be understood that the forthcoming example description is not intended to be limiting and that variations and adaptations thereof may be implemented without departing from the intended scope of the present disclosure.

According to the present example implementation, given an EKG time series s_{EKG}(t) with the temporal index t, instants are first detected corresponding to the peak timing of the QRS complex and these instants are taken as the temporal references for individual cardiac cycles. A mapping f(t) may then be established to transform the temporal index t to the m^{th} cardiac cycle with a latency φ:

$$f \left(t\right) \to \left\{m , φ\right\} , m = 1 , ⋯ , {m}_{m a x}$$

Thus, s_{EKG}(t) was mapped to its cardiac cycle m and latency φ according to

$${s}_{E K G} \left(t\right) \to {s}_{E K G}^{m} \left(φ\right) .$$

In analogous fashion, the mapping f(t) also allows a given EEG signal at the temporal index t s_{EEG}(t) to be related to the m^{th} cardiac cycle with a latency φ

$${s}_{E E G} \left(t\right) \to {s}_{E E G}^{m} \left(φ\right) .$$

An e-dimensional manifold of cardiac dynamics may be created from time-lagged (temporally shifted) samples at multiples of latency (time lag or temporal shift) _{T} of the EKG:

$${k}_{E K G}^{m} \left(φ\right) = \left[{s}_{E K G}^{m} \left(φ\right) , {s}_{E K G}^{m} \left(φ + τ\right) , {s}_{E K G}^{m} \left(φ + \left(e - 1\right) τ\right)\right] ,$$

where _{T} denotes the latency within a cardiac cycle. A matrix K_{EKG}(φ) may then be created by vertically concatenating

$${k}_{E K G}^{m} \left(φ\right)$$

across cardiac cycles:

$${\text{K}}_{\text{EKG}} \left(φ\right) = {\left[{k}_{E K G}^{1} \left(φ\right) ; {k}_{E K G}^{m} \left(φ\right) ; . … , {k}_{E K G}^{{m}_{m a x}} \left(τ\right)\right]}^{\text{T}} ,$$

where the superscript T denotes the matrix transpose.

For the EKG at cycle m, it is then possible to search across cardiac cycles to identify n similar cardiac cycles that are in the proximity of

$${k}_{E K G}^{m} \left(φ\right)$$

in the K_{EKG}(φ) manifold. The “neighboring” (e.g. locally similar) cardiac cycles may be indexed by mi, i = 1 , ..., n; n ≤ mₘₐₓ. In the K_{EKG}(φ) manifold, the distance d^{mᵢ} between

$${k}_{E K G}^{m} \left(φ\right)$$

and

$${k}_{E K G}^{{m}_{i}} \left(φ\right)$$

may be determined, for example, by the Euclidean distance. All n cardiac cycles in the vicinity of the m^{th} cardiac cycle may then be identified, and optionally ranked, e.g., in descending order: d^{m₁} ≥ d^{m₂} ≥ ··· ≥ d^{mₙ}.

A linear combination of EEG signals in similar cardiac cycles {mᵢ} (i = 1 , ..., n) with the latency φ may then employed to develop a model of the EEG signal,

$${\hat{s}}_{E E G}^{m} \left(φ\right) ,$$

in the m^{th} cardiac cycle with the latency φ, as follows:

$${\hat{s}}_{E E G}^{m} \left(φ\right) = {\sum }_{i = 1}^{n} {w}_{i} {s}_{E E G}^{{m}_{i}} \left(φ\right) .$$

In a manner suggested by the prediction procedure in modeling dynamics in a manifold, the weightings wᵢ may be calculated, for example, by:

$${u}_{i} = {e}^{- {d}^{{m}_{i}}} / {e}^{- {d}^{{m}_{1}}} ,$$

$${w}_{i} = {u}_{i} / \sum {}_{i = 1}^{n} {u}_{i} .$$

Briefly, the weighting coefficients wᵢ and data

$${s}_{E E G}^{{m}_{i}} \left(φ\right)$$

to model the EEG measurement

$${s}_{E E G}^{m} \left(φ\right)$$

by

$${\hat{s}}_{E E G}^{m} \left(φ\right)$$

are based on the similarity between the representations of

$${s}_{E E G}^{m} \left(φ\right)$$

and

$${s}_{E E G}^{{m}_{i}} \left(φ\right)$$

in the cardiac dynamics in K_{EKG}(φ).

Although alternative interpolation methods may also work in DMH, for the sake computational simplicity, the present example is limited to a linear interpolation method to model

$${s}_{E E G}^{m} \left(φ\right) \text{by} {\hat{s}}_{E E G}^{m} \left(φ\right) .$$

The modeled EEG

$${\hat{s}}_{E E G}^{m} \left(φ\right)$$

can be transformed to the temporal index t by a mapping

$${\hat{s}}_{E E G}^{m} \left(φ\right) \to {\hat{s}}_{E E G} \left(t\right) .$$

Because ŝ_{EEG}(t) was linearly interpolated from the EEG recording with the same cardiac latency in other cardiac cycles, it ideally contains only BCG features without other physiological information specific to time instant t. If the effect of interest occurred asynchronously to cardiac cycles, then subtracting ŝ_{EEG}(t) from s_{EEG}(t) yielded

$${S}_{E E G}^{B C G -} \left(t\right)$$

as the EEG signal after BCG artifact suppression:

$${S}_{E E G}^{B C G -} \left(t\right) = {S}_{E E G} \left(t\right) - {\hat{S}}_{E E G} \left(t\right) .$$

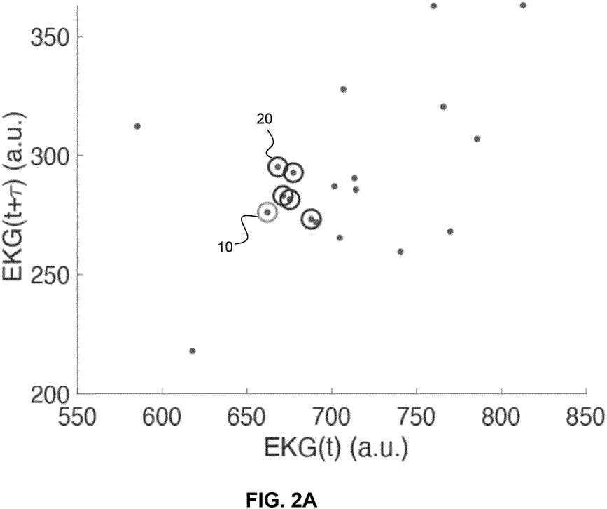

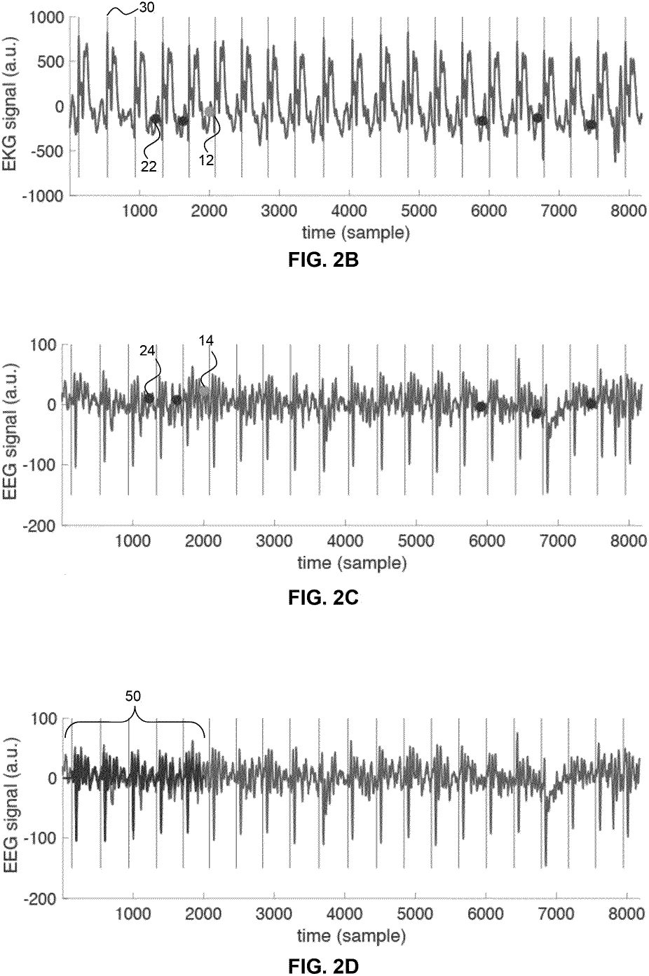

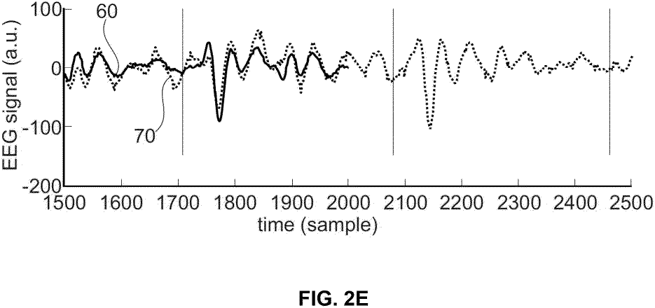

FIGS. 2A-2E provide an illustration of the dynamic modeling of heartbeats approach to suppress the BCG artifacts in EEG. In FIG. 2A, a two-dimensional manifold (e = 2) of cardiac dynamics is shown. Each dot indicates one cardiac cycle. In the present and non-limiting 2D manifold example, a pair of EKG signals at its QRS peak and a delay _{T} ₌ 2 ms is employed to represent one cardiac cycle. The dimension of the manifold e and the delay τ may be selected to suitably characterize the waveform features. For example, the skilled artisan may experiment with different values of these parameters in order to achieve a suitable and/or desired level of performance in BCG artifact rejection. The dot 10 with a light gray circle indicates the selected cardiac cycle of interest. Five nearest cardiac cycles on this 2D manifold are indicated by dark gray circles 20.

In FIGS. 2C and 2D, EKG and EEG waveforms are respectively plotted. The vertical lines 30 indicate the detected QRS peaks. The light gray (12, 14) and dark gray (22, 24) dots correspond to the analogous cardiac cycles identified on the 2D manifold in FIG. 2A.

FIG. 2D plots the recorded and interpolated EEG waveforms based on the EEG signals at five cardiac cycles with the 2D manifold representation nearest to that of the current one. Because of the similar cardiac cycles, both recorded and interpolated waveforms are expected to have similar BCG artifacts.

In FIG. 2E, details of the recorded (70, s_{EEG}(t)) and interpolated (60, ŝ_{EEG}(t)) EEG waveforms show reasonable estimates of the EEG signal with clear BCG artifacts.

It will be understood that the preceding example methods may be adapted without departing from the intended scope of the present disclosure. For example many different methods may be employed to identify cardiac cycles. For example, while the present inventor employed the Pan-Tompkin method to identify cardiac QRS complexes, which are taken as the temporal reference, other methods may be additionally or alternatively employed to identify and define the onset, duration, and phase of cardiac cycles.

Furthermore, while the example implementations described herein employs the use of a two-dimensional manifold (two latencies within a cardiac cycle) to define an instantaneous EKG signal, alternative implementations may be employed to construct a suitable manifold. In particular, the representation of the EKG signal in time or other transformed domains, the number of latencies, the temporal separation between latencies can be varied for other representations of EKG dynamics.

It is also noted that alternative methods may be employed to for the identification and interpolation of EEG waveforms with similar cardiac dynamics. In the aforementioned non-limiting example implementation, five EKG cycles were employed that exhibited similar cardiac dynamics based on the Euclidean distance in a two-dimensional manifold to model the EEG waveform by linear combining similar EEG waveforms with weights in proportion to the inverse of the distance. However, it will be understood that other distance measures on the cardiac cycle manifolds and methods of combining cardiac cycles based on the distance measures can be used.

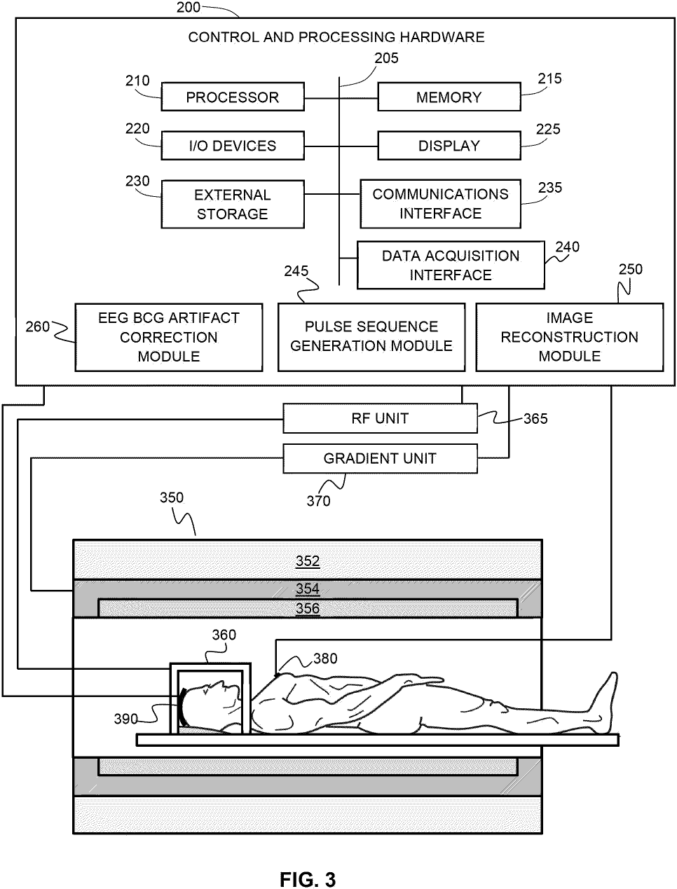

Referring now to FIG. 3, an example system is illustrated for performing suppression of BCG artifacts in EEG data using dynamic heart modeling based on the processing of EKG data. While the example embodiments of the present disclosure may be adapted to a wide range of implementations, the example system shown in FIG. 3 includes a magnetic resonance imaging (MRI) scanner 350. The MRI scanner 350 employs a main magnet 352 to produce a static magnetic field B0 that generates a polarization in the patient, and gradient coils 54 for generating magnetic field gradients. While a body coil 356 may be employed to generate RF energy and detect RF signals from the patient, the present example implementation is shown employing a head coil 360. The RF pulses are generated by an RF unit 365, and the magnetic field gradients are generated by a gradient unit 370.

As shown in the figure, one or more EKG sensors 380 contact the patient for the detection of EKG signals, and a MRI-compatible EEG electrode array 390 is shown contacting the head of the patient.

In the example system shown in FIG. 3, control and processing hardware 200 controls the MRI scanner to generate RF pulses according to a suitable pulse sequence. The control and processing hardware 200 may include, for example, one or more processors 210, memory 215, a system bus 205, one or more input/output devices 220, and a plurality of optional additional devices such as communications interface 235, data acquisition interface 240, display 225, and external storage 230. The control and processing hardware 200 is interfaced with the MRI scanner 350 for controlling the acquisition of the received MRI signals. The control and processing hardware 200 acquires the received MRI signals from the RF unit 365 and processes the MRI signals in order to perform image reconstruction and generate MRI images.

The control and processing hardware 200 is also operatively coupled to the EKG sensor 380 and the EEG electrode array 390 and thus receives and processes the EKG and EEG signals.

The control and processing hardware 200 may be programmed with a set of instructions which when executed in the processor causes the system to perform one or more methods described in the present disclosure. For example, as shown in FIG. 3, control and processing hardware 200 may be programmed with instructions in the form of a set of executable processing modules that include, for example, an EEG BCG artifact suppression module 260 for implementing the example embodiments described above, an MRI pulse sequence generation module 245 and an MRI image reconstruction module 250.

The pulse sequence generation module 245 may be implemented using algorithms known to those skilled in the art for pulse sequence generation. During MRI scanning, RF data is received from the RF coils 360/356. The pulse sequence generation module 245 establishes the sequence of RF pulses and magnetic field gradients depending on the desired imaging sequence, MR signals responsively emitted by the patient and detected by the coils 360/356 are acquired. The image reconstruction module 245 processes the acquired MRI signals to perform image reconstruction and MRI image generation.

It is to be understood that the example system shown in FIG. 3 is illustrative of a non-limiting example embodiment, and is not intended to be limited to the components shown. For example, the system may include one or more additional processors and memory devices. Furthermore, one or more components of control and processing hardware 200 may be provided as an external component that is interfaced to a processing device.

Some aspects of the present disclosure can be embodied, at least in part, in software, which, when executed on a computing system, configures the computing system as a specialty-purpose computing system that is capable of performing the signal processing and noise reduction methods disclosed herein, or variations thereof. That is, the techniques can be carried out in a computer system or other data processing system in response to its processor, such as a microprocessor, CPU or GPU, executing sequences of instructions contained in a memory, such as ROM, volatile RAM, non-volatile memory, cache, magnetic and optical disks, cloud processors, or other remote storage devices. Further, the instructions can be downloaded into a computing device over a data network, such as in a form of a compiled and linked version. Alternatively, the logic to perform the processes as discussed above could be implemented in additional computer and/or machine readable media, such as discrete hardware components as large-scale integrated circuits (LSI’s), application-specific integrated circuits (ASIC’s), or firmware such as electrically erasable programmable read-only memory (EEPROM’s) and field-programmable gate arrays (FPGAs).

A computer readable medium can be used to store software and data which when executed by a data processing system causes the system to perform various methods. The executable software and data can be stored in various places including for example ROM, volatile RAM, non-volatile memory and/or cache. Portions of this software and/or data can be stored in any one of these storage devices. In general, a machine-readable medium includes any mechanism that provides (i.e., stores and/or transmits) information in a form accessible by a machine (e.g., a computer, network device, personal digital assistant, manufacturing tool, any device with a set of one or more processors, etc.).

Examples of computer-readable media include but are not limited to recordable and non-recordable type media such as volatile and non-volatile memory devices, read only memory (ROM), random access memory (RAM), flash memory devices, floppy and other removable disks, magnetic disk storage media, optical storage media (e.g., compact discs (CDs), digital versatile disks (DVDs), etc.), network attached storage, cloud storage, among others. The instructions can be embodied in digital and analog communication links for electrical, optical, acoustical or other forms of propagated signals, such as carrier waves, infrared signals, digital signals, and the like. As used herein, the phrases “computer readable material” and “computer readable storage medium” refer to all computer-readable media, except for a transitory propagating signal per se.

In addition to improving the EEG-MRI data from healthy participants, the present example embodiments may be adapted to assist clinicians in detecting interictal spikes (IIS) from concurrent EEG-fMRI of epilepsy patients. In such clinical applications, detecting IIS occurrences is the crucial step for subsequent analysis of fMRI data. Improving the EEG quality may improve the sensitivity and specificity of spike detection and subsequent fMRI-based delineation of the irritative zones, thus assisting neurosurgical decision-making.

The following examples are presented to enable those skilled in the art to understand and to practice embodiments of the present disclosure. They should not be considered as a limitation on the scope of the disclosure, but merely as being illustrative and representative thereof.

In the Examples provided below, example implementations of the present BCG subtraction embodiments are presented, with empirical results, in which DMH is employed to suppress BCG artifact in EEG data recorded concurrently with MRI. Specifically, DMH performance in identifying evoked neuronal responses is benchmarked with respect to the OBS method. The performance of the example implementations of the DMH systems and methods disclosed herein was tested and specifically compared with the Optimal Basis Set (OBS) method on EEG data recorded inside a 3T MRI system with either no MRI acquisition (Inside-MRI), echo-planar imaging (EPI-EEG), or fast MRI acquisition using simultaneous multi-slice and inverse imaging methods (SMS-Inl-EEG). In a steady-state visual evoked response (SSVEP) paradigm, the transient response was 88% higher when the DMH method was used in comparison to OBS in the Inside-MRI condition. The 15-Hz oscillatory neuronal activity at the visual cortex after DMH processing was about 130% of that achieved by OBS processing for Inside-MRI, SMS-Inl-EEG, and EPI-EEG conditions. The sensitivity and specificity of detecting the 15-Hz SSVEP were consistently higher by DMH than OBS in the three experimental conditions.

A substantial improvement in BCG artifact suppression was therefore demonstrated in the present example experimental studies. The DMH process thus appears to provide improved BCG suppression in EEG signal, relative to known methods. The present results motivate the application of DMH to neuroscience and clinical applications of concurrent EEG-MRI experiments to obtain high-quality concurrent measurements of electrophysiological and hemodynamic responses, particularly in cases where the MRI gradient artifact over EEG is effectively suppressed.

Moreover, in addition to providing a performance improvement, the present DMH embodiments improve the functioning of an EEG processing system by providing a more computationally efficient method suppressing BCG artifacts. Future implementations may also assist in improving the quality of EEG data recorded in high-field MRI systems for neuroscientific and clinical applications.

### EXAMPLES

### Example 1: Experimental Design and Measurement

Thirty healthy control participants (age: 21 - 45 years; 18 female) gave written free and informed consent before participating in the study. Nine participated in the evoked response experiment. Part of the visual evoked response data was analyzed in a previous study of fast MRI and EEG conducted in the laboratory.

The experiments involved recording evoked EEG responses in various MRI acquisition environments. The measurement of evoked responses involved steady-state visual evoked potentials (SSVEPs). Specifically, participants were instructed to fixate visually on a crosshair at the center of the display screen. To ensure that participants maintained visual fixation, they were instructed to press a button when the crosshair changed color from black to red. The red crosshair appeared for 1 s randomly throughout the experiment. Asynchronous with the crosshair stimulus, flashing checkerboard patterns (flashing frequency = 7.5 Hz, stimulus duration 1 s) were also presented randomly with a minimal inter-stimulus interval of 2 s to elicit SSVEPs. The checkerboard subtended 4.3° of visual angle and contained 24 evenly distributed radial wedges with eight concentric rings of equal width. The 7.5-Hz flashing checkerboard stimuli were expected to elicit a strong SSVEP with a frequency of 15 Hz. Onsets of checkerboard flashing were temporally jittered between 0.2 s and 0.9 s after the beginning of each MRI acquisition of the brain volume to minimize artifacts caused by MRI gradient coil switching in concurrent fast fMRI and EEG acquisition (see details below). Three six-minute runs were collected for each condition from each participant.

All MRI data were measured on a 3T MRI scanner using a 64-channel head-neck receiver coil array (Prisma or Skyra, Siemens, Erlangen, Germany) with a hole at the vertex of the head coil for routing the EEG cables. Structural images were acquired with the magnetization-prepared rapid gradient echo (MPRAGE) pulse sequence (repetition time TR=2530 ms, echo time TE = 3.03 ms, isotropic voxel resolution = 1 mm, field of view FOV = 256 mm, flip angle = 7°, matrix size = 224×256, generalized auto-calibrating partial parallel acquisition (GRAPPA) acceleration factor = 2). As EEG recorded inside MRI suffers from artifacts caused by MRI gradient coil switching and heartbeats, we took EEG with two different kinds of fMRI to probe the performance of BCG artifact suppression with low and high levels of residual gradient artifacts. Specifically, functional images were acquired with a fast fMRI sequence (simultaneous multi-slice inverse imaging (SMS-Inl); TR/TE = 2000/30 ms, FOV=210 mm, flip angle = 30°, resolution = 5x5x5 mm³, slice numbers = 24). Spatial encoding was performed in 0.1 s, leaving 1.9 s (95 % of the TR interval) free from MRI gradient coil operation. We previously showed that concurrent temporally sparse fast fMRI and EEG gave much reduced gradient artifact. For comparison, T₂^{*}-weighted echo-planar imaging (EPI) was also acquired for fMRI with a typical spatiotemporal resolution (TR/TE = 2000/36 ms, FOV = 224 mm, flip angle = 90°, number of slices = 30, resolution = 3.5 mm x 3.5 mm x 4 mm, GRAPPA acceleration = 2).

EEG was recorded inside the 3T MRI scanner using an MRI-compatible system (BrainAmp MR Plus, Brain Products, Gilching, Germany) with a 32-channel EEG cap (BrainCap MR, Brain Products, Gilching, Germany). Electrodes were arranged following the 10-20 international standard. The EEG data were referenced with respect to the FCz electrode, with the ground reference taken at the AFz electrode. The electrocardiogram (EKG) was also measured by placing an electrode on the back of the participant.

To ensure highly synchronous EEG and EKG recordings with respect to the fMRI acquisitions, an established procedure was adopted using a frequency divider and phase-locking device as part of the EEG system (BrainAmp MR Plus, Brain Products, Gilching, Germany). The phase-locking device received the 10 MHz transistor-to-transistor logic (TTL) signal from the clock board of the MRI system via a coaxial cable and produced a 5-kHz output signal to synchronize the EEG acquisition. The MRI TR value recorded by the EEG system was confirmed to match the prescribed TR value at the MRI console with 5 \-kHz sampling rate. The impedance of each electrode was verified as < 9 kΩ (including the built-in 5 kΩ impedance) after applying conductive gel. The EEG cap wire bundle was straightened and fixed along the main magnetic field for 50 cm and connected to an EEG amplifier at the rear of the magnet (just outside the bore) to reduce artifacts generated by the wire. The positions of electrodes over the scalp of a participant were measured by a digitizer (Fastrak, Polhemus, Vermont, Canada). These positions were used to register EEG electrodes with the head model derived from structural MRI.

EEG was measured separately in three different conditions. EPI-EEG and SMS-Inl-EEG denotes the concurrent recording of EEG with EPI and SMS-Inl, respectively. In addition, EEG was recorded inside the MRI system without imaging (Inside-MRI) providing a condition that yielded EEG signals with BCG artifact but no gradient artifact (GA).

### Example 2: EEG Data Analysis

EEG processing was implemented in MATLAB (Mathworks, Natick, MA, U.S.A). For EPI-EEG and SMS-Inl-EEG, GA was suppressed using the average artifact subtraction (AAS) method. To account for the timing difference in the clock accuracy between MRI (10 MHz) and EEG (5 kHz) systems, further alignment between the GA template and the EEG data was achieved by interpolating with an accuracy of 0.2, 0.02, 0.002 and 0.0002 samples in four iterations to achieve numerical sampling rates of 0.025 MHz, 0.25 MHz, 2.5 MHz, and 25 MHz, respectively. The GA template was dynamically estimated over seven TR intervals. The EEG data were further zero-phase band-pass filtered between 1 Hz and 50 Hz, and down-sampled to 500 Hz.

### Example 3: BCG Artifact Suppression

BCG artifacts were suppressed according to the present example method based on dynamic heart modeling as described above, and artifact suppression was also performed, for comparison, using the optimal basis set (OBS) method. As suggested by previous studies on OBS, the order of the Principal Component Analysis was chosen to be three.

Subsequently, the SSVEPs were calculated by first extracting EEG signals between 200 ms before and 1000 ms after the onset of each visual stimulus for all trials of a given measurement (EPI-EEG, SMS-Inl-EEG, and inside-MRI). For all EEG trials, the constant and the linear drift were removed by linear regression. Spurious trials with a maximum EEG signal >700 µV were excluded. The SSVEPs were then derived by averaging across trials at each electrode. Oscillatory features in the evoked EEG signals were quantified using the Morlet wavelet transform with a temporal window of 5 cycles. The 15-Hz oscillations were then investigated for the SSVEPs.

### Example 4: EEG Source Modeling

The scalp EEG data and the neuronal current sources at time t were related to each other by the linear equation:

$$\text{y} \left(t\right) = \text{A} \text{x} \left(t\right) + \text{n} \left(t\right) ,$$

where y(t) denoted the collection of EEG data across electrode contacts, x(t) denoted the neuronal current strength, and n(t) denoted the contaminating noise, and A was the lead field matrix. Specifically, for a unit current dipole source at location r in the +x, +y, or +z direction, the electric potentials measured at all electrode contacts were:

$$\text{a} \left(\text{r}\right) = \left[{\text{a}}_{\text{x}} \text{} \left(\text{r}\right) , {\text{a}}_{\text{y}} \text{} \left(\text{r}\right) , {\text{a}}_{\text{z}} \text{} \left(\text{r}\right)\right] .$$

Assembling a(r) across all possible current dipole source locations created the lead field matrix A:

$$\text{A} = \left[\text{a} \left({\text{r}}_{\text{1}}\right) , \text{a} \left({\text{r}}_{\text{2}}\right) , ... , \text{a} \left({\text{r}}_{k}\right) , ...\right] , k = 1 , .... , d .$$

where d denotes the total number of current dipole source locations. rₖdenotes the location of the k^{th} current dipole source location.

The lead field matrix A was created from the T₁-weighted MPRAGE MRI data using scalp, skull, and brain models generated by FreeSurfer (https://surfer.nmr.mgh.harvard.edu). Potential EEG source locations at the gray and white matter boundary were identified with approximately 5-mm separation between the nearest neighboring source locations. The locations of EEG electrodes were manually registered to the scalp model. The matrix A was then calculated by the OpenMEEG package (https://openmeeg.github.io/). The relative conductivity values for air, scalp, brain parenchyma, and skull were set as 0, 1, 1, and 0.0125, respectively.

The Minimum-Norm Estimate (MNE) was used to estimate x(t):

$${x}_{M N E} \left(t\right) = R {A}^{T} {\left(A R {A}^{T} + λ C\right)}^{- 1} y \left(t\right) ,$$

where R was the source covariance matrix and C was the noise covariance matrix:

$$C = \left〈n \left(t\right) {n}^{T} \left(t\right)\right〉 ,$$

with the operator 〈▪〉 taking the ensemble average across realizations. In practice, C was estimated from y(t) during the pre-stimulus interval (from -200 ms to 0 ms) with data concatenated across trials in the SSVEP measurements. The regularization λ tuned the balance between the strength of the estimated neural current strength and the discrepancy between the modeled and measured data. The value λ = 10 was used in this study as suggested by previous work.

The spatial distribution of estimated neuronal currents at each time instant from each participant was then spatially registered to an arbitrarily selected individual: “fsaverage” in the FreeSurfer library. This registration was done via a spherical coordinate system. The neuronal currents were averaged across participants for each condition separately. The significance of the neuronal current distribution was estimated at each source location by calculating the ratio between the instantaneous value and the standard deviation calculated over the baseline interval. These ratios constituted dynamic statistical parametric maps (dSPMs) and were reported to follow a t-like distribution. To model the oscillatory neuronal current distributions in the brain, EEG signals were first filtered by the Morlet wavelet transform and then modeled by the MNE. The filtered coefficients were then used to generate the MNE and dSPMs.

### Example 5: Performance Measures

The performance of BCG artifact suppression was measured for the SSVEP experiments by first characterizing the difference of 15-Hz oscillatory responses, for scalp EEG at O1, O2, and Oz electrodes over the interval of +0.2 s and 1.0 s after the visual stimulus onset. The electrode topology and time interval were chosen based on practice in previous SSVEP studies. The SSVEP waveforms were also characterized in terms of their total power, transient response, and oscillatory response. The detection of neuronal sources for SSVEPs was also compared between OBS and DMH methods for the three MRI conditions (Inside-MRI, EPI-EEG, SMS-Inl-EEG) using Receiver Operating Characteristic curves, where the boundary of the primary visual cortex was delineated from an independent structural MRI and fMRI studies. The true-positive and false-negative detection was taken as the areas intersecting the anatomically defined primary visual cortex and the brain area showing significant and insignificant 15-Hz oscillations, respectively. The true-negative and false-positive detection was taken as the areas intersecting the brain area outside the anatomically defined primary visual cortex and the brain area showing insignificant and significant 15-Hz oscillations, respectively.

### Example 6: Results

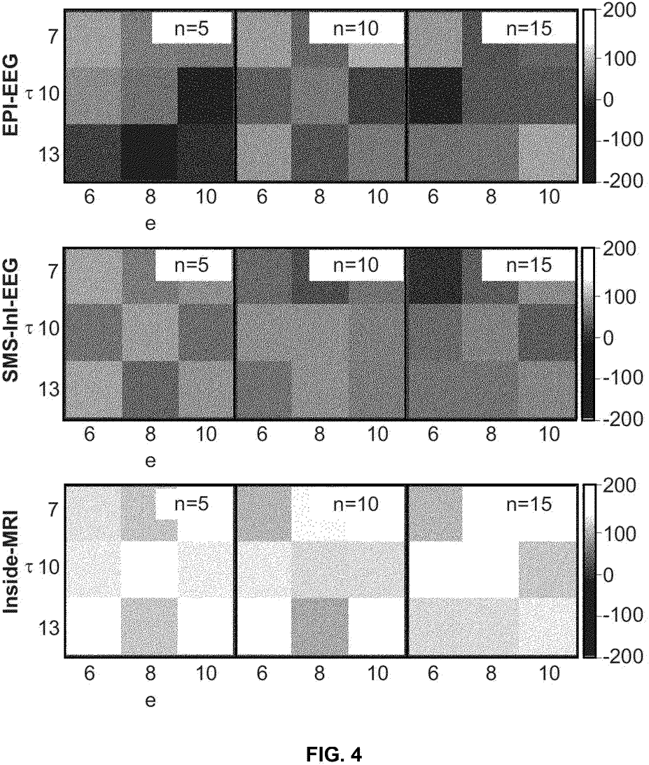

FIG. 4 shows the post-stimulus 15-Hz oscillatory power difference between SSVEP results using DMH and OBS to suppress BCG artifacts. These differences were calculated for different combinations of DMH parameters (n = 5, 10, or 15; e = 6, 8, or 10; τ = 7, 10, or 13), for EEG collected concurrently with EPI (EPI-EEG), SMS-Inl (SMS-Inl-EEG), and inside the magnet without MRI (Inside-MRI). Except for a few cases in EPI-EEG, DMH generally improved the detection of post-stimulus 15-Hz oscillation of the averaged EEG across O1, O2, and Oz electrodes by showing positive power differences for DMH compared to OBS regardless of MRI condition (EPI-EEG, SMS-Inl-EEG, or Inside-MRI). This suggested that DMH outperformed OBS stably among parameter combinations. Subsequently, all reported numbers for DMH are provided with the parameter combination of (ε = 8, τ= 10, and n = 10) which reasonably approximate the average performance of the method.

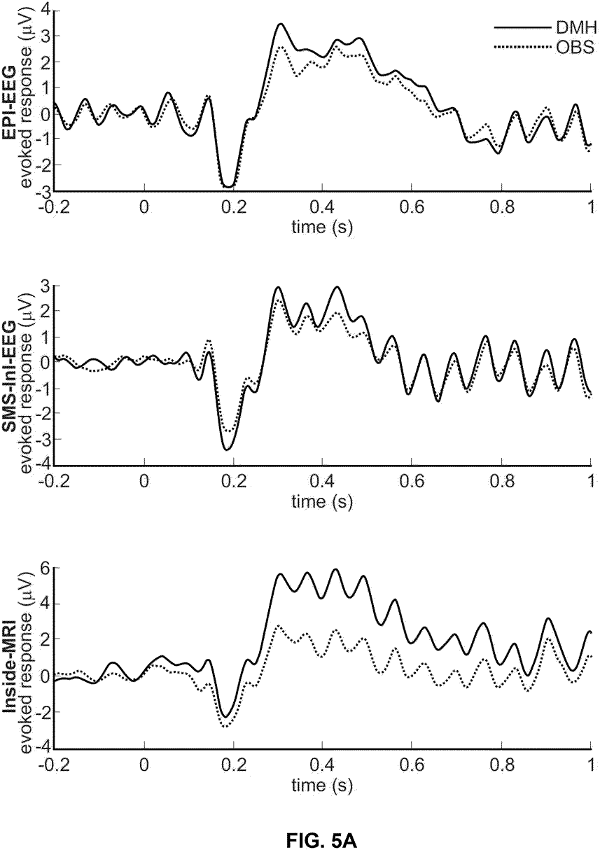

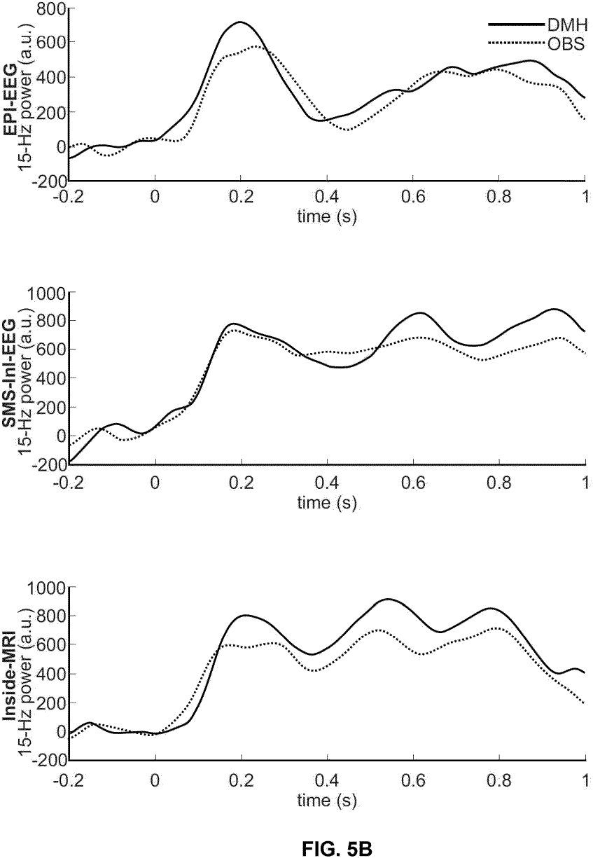

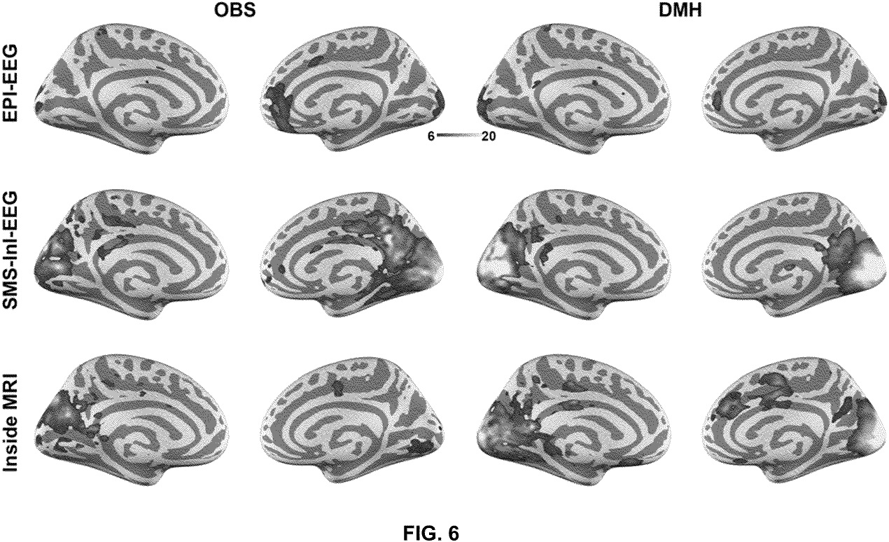

FIG. 5A shows the averaged EEG traces across O1, O2, and Oz electrodes using OBS or DMH to suppress BCG artifacts. The averaged EEG power after the stimulus onset was found to be consistently higher after using DMH to suppress BCG artifacts in comparison to using OBS, regardless of whether EEG was recorded concurrently with EPI (EPI-EEG; OBS: 1.73 +/- 0.03 µV², DMH: 2.55 +/- 0.04 µV²; two-sample test: t = 13.76, p <10⁻¹⁰), SMS-Inl(SMS-Inl-EEG; OBS: 1.08 +/- 0.02 µV², DMH: 1.78 +/- 0.04 µV²; two-sample test: t = 13.51, p <10⁻¹⁰), or inside the magnet without MRI (Inside-MRI; OBS: 1.51 +/-0.03 µV², DMH: 8.64 +/- 0.14 µV²; two-sample test: t = 54.32, p <10⁻¹⁰). This amounted to a 48 % (2.55/1.73 - 1), 65 % (1.78/1.08 - 1), and 472% (8.64/1.51 -1) gain in the averaged EEG power for EPI-EEG, SMS-Inl-EEG, and Inside-MRI, respectively.

FIG. 5B shows the 15-Hz time-frequency representation (TFR) of the EEG waveform averaged across O1, O2, and Oz electrodes. For EPI-EEG, the difference between waveforms after OBS and DMH suppressing BCG artifacts was not visually clear. This difference was clearer for SMS-Inl-EEG after +500 ms. For Inside-MRI, the 15-Hz TFR was consistently stronger with DMH than with OBS after +200 ms. Two-sample t-tests were performed to quantify the TFR difference between OBS and DMH processing. All conditions show significantly larger 15-Hz TFR by DMH than OBS (EPI-EEG: OBS: 326.24 +/- 2.16, DMH: 378.38 +/- 2.27; t = 14.73, p <10⁻¹⁰; SMS-Inl-EEG: OBS: 566.64 +/- 2.16, DMH: 627.65 +/- 2.77; t = 17.38, p <10⁻¹⁰; Inside-MRI: OBS: 497.94 +/- 2.50, DMH: 606.68 +/- 3.47; t = 25.41, p <10⁻¹⁰).

The neuronal current distributions of the 15-Hz SSVEP were subsequently examined. FIG. 6 shows the average (calculated over the time window from +200 ms to +1000 ms) ratio of the power of the 15-Hz time-frequency representation with respect to that in the pre-stimulus interval. The difference between OBS and DMH was not readily apparent in the EPI-EEG maps. Both maps showed the expected high-power ratios around the posterior occipital lobe with intriguing signals around the right frontal pole. For SMS-Inl-EEG, the difference between OBS and DMH was clear: a much stronger power ratio of 15-Hz oscillation was found around the visual cortex in DMH than OBS. This performance was similarly observed for the Inside-MRI results. The DMH method yielded a stronger and more balanced 15-Hz power ratio between left and right visual cortices than the OBS method. A strong power ratio around the cingulate cortex was found in both OBS and DMH. This may be due to the supplementary eye field activity to suppress saccades to fixate at the center of the visual field and ignore the flickering visual stimuli at the periphery of the visual field.

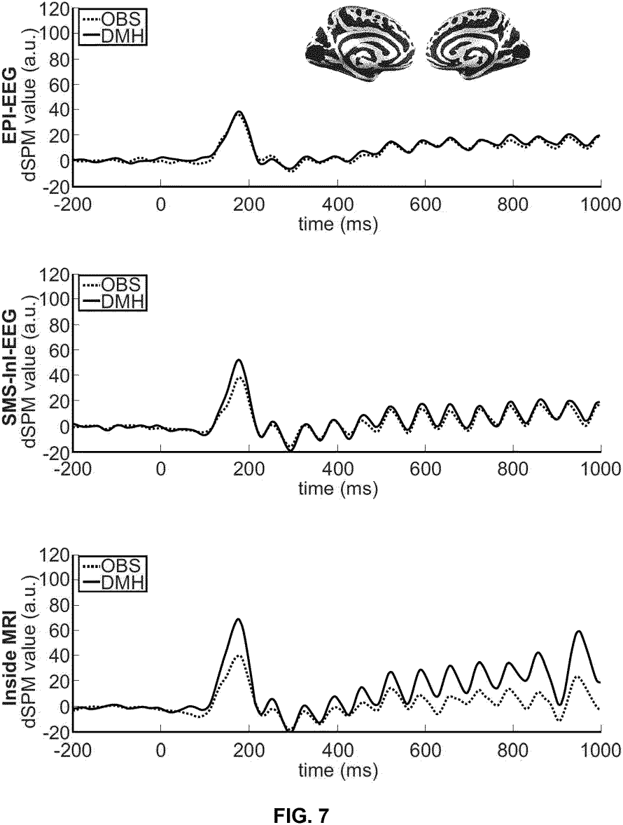

FIG. 7 shows the time courses of the estimated neuronal currents at the visual cortex in three conditions after either OBS or DMH suppressing BCG artifacts. All time courses exhibited a transient response at around 200 ms, followed by oscillatory activities. In EPI-EEG, the transient response and the following oscillatory activities were visually similar between DMH and OBS. In SMS-Inl-EEG, a stronger transient response was found with DMH than OBS (56 for DMH and 41 for OBS; 36% gain). Visually clear 15-Hz oscillation was found in data processed with both methods. DMH visually outperformed OBS in Inside-MRI by a much stronger transient response (105 for DMH and 56 for OBS; 88% gain) and 15-Hz oscillation (see quantification next).

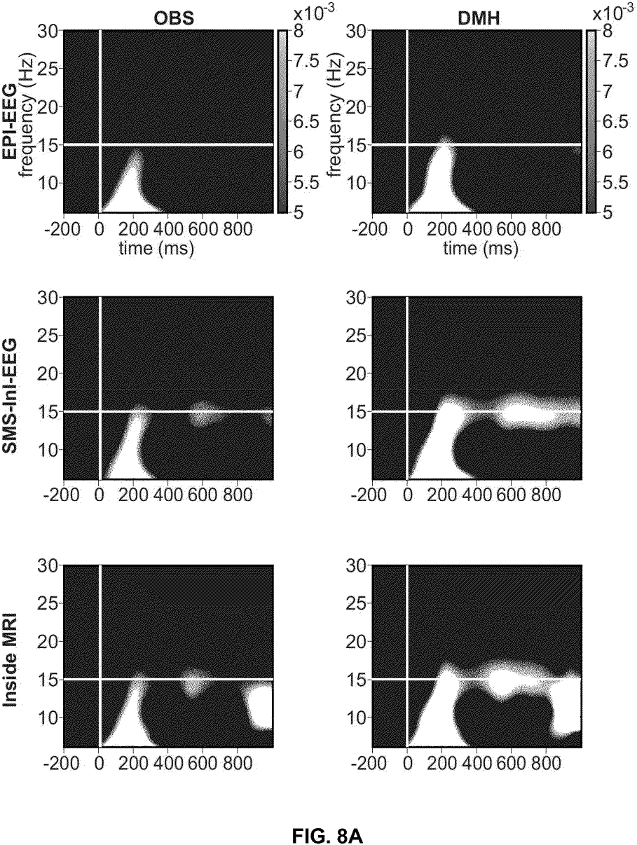

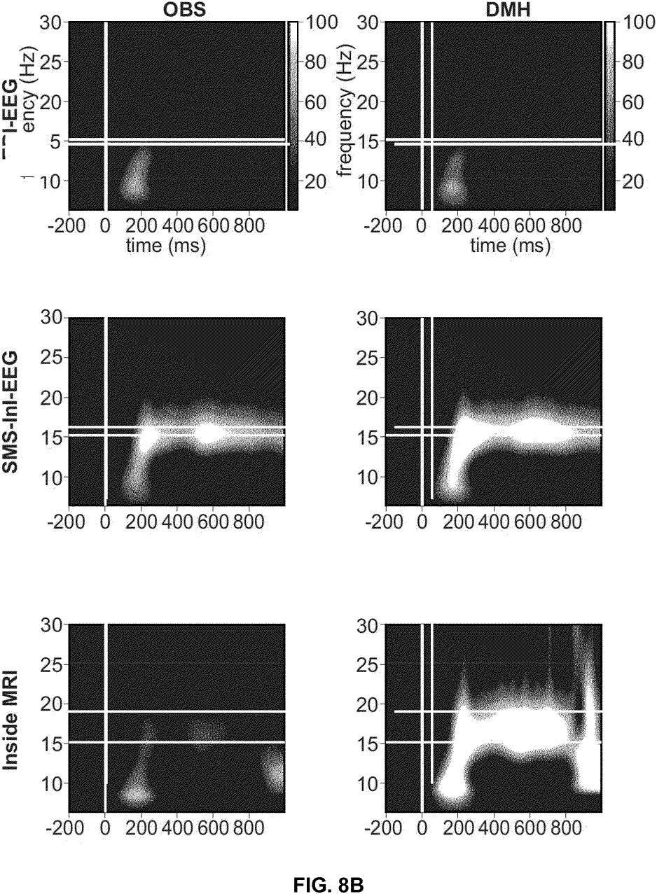

FIG. 8A shows the TFRs of the estimated neuronal current at the visual cortex. Slightly stronger 15-Hz oscillation was inspected in EPI-EEG using DMH than OBS. The 15-Hz oscillation differences were visually clearer in SMS-Inl-EEG and Inside MRI conditions. The 15-Hz oscillation between +0.2 s and +1.0 s after the stimulus onset for DMH and OBS were 4.99 x 10⁻³ ± 8.16 x 10⁻⁴ and 3.88 x 10⁻³ ± 7.68 x 10⁻⁴, respectively. DMH offered significantly stronger 15-Hz oscillation (29% gain; two-sample t-test t = 19.83; p < 1 x 10⁻²⁰). In SMS-Inl-EEG, the 15-Hz oscillation between +0.2 s and +1.0 s after the stimulus onset for DMH and OBS were 7.57 x 10⁻³ ± 7.24 x 10⁻⁴ and 5.79 x 10⁻³ ± 6.35 x 10⁻⁴, respectively. DMH offered significantly stronger 15-Hz oscillation (31% gain; two-sample t-test t = 36.86; p < 1 x 10⁻²⁰). In Inside-MRI, the 15-Hz oscillation between +0.2 s and +1.0 s after the stimulus onset for DMH and OBS were 7.62 x 10⁻³ ± 7.24 x 10⁻⁴ and 5.79 x 10⁻³ ± 5.88 x 10⁻⁴, respectively. DMH also offered significantly stronger 15-Hz oscillation (32% gain; two-sample t-test t = 39.09; p < 1 x 10⁻²⁰);

Noise-normalized TFRs were also calculated for each data set, where the noise level was separately estimated by the standard deviation of signals in the pre-stimulus interval at each frequency (FIG. 8B). Ratios were taken between post-stimulus instants and the estimated noise level. Results with DMH and OBS were similar in the time-frequency representation between 5 Hz and 30 Hz with similar 15-Hz dynamics in EPI-EEG. The average 15-Hz noise-normalized TFR was 11.07+/-3.81 and 9.81+/-3.51 for OBS and DMH, respectively. The difference was not significant. DMH shows stronger and persistent noise-normalized 15-Hz oscillation than OBS in SMS-Inl-EEG. Quantitatively, the average 15-Hz TFR was 83.94+/-14.52 and 103.59+/-21.72 for OBS and DMH, respectively. The difference between the two methods was significant (two-sample t-test; p <0.001). This difference was significant for the EEG data recorded during the Inside-MRI condition, where the strongest noise-normalized 15-Hz oscillation was found. In this case, the noise-normalized 15-Hz TFR was 25.16+/-5.36 and 111.12+/-23.05 for OBS and DMH, respectively (two-sample t-test; p < 0.0001). Note that the noise-normalized 15-Hz oscillation for inside-MRI was smaller than SMS-Inl-EEG when using OBS (left middle and bottom panels; FIG. 8B). This was likely attributed to the difference in baseline noise level between SMS-Inl-EEG and Inside-MRI, because of their similar strength without noise normalization (left middle and bottom panels; FIG. 8A).

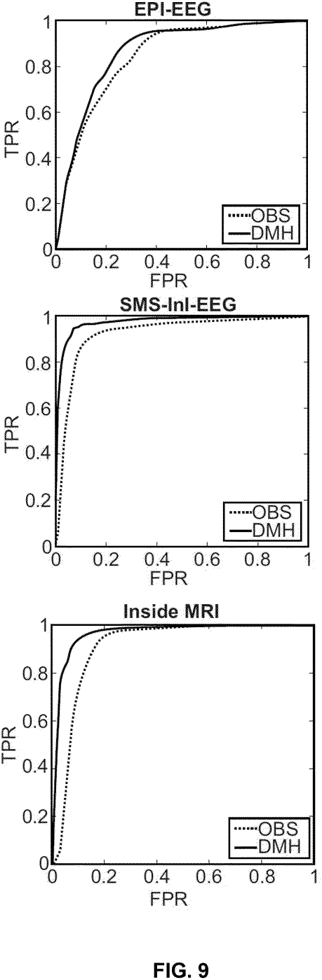

Based on the boundary of the primary visual cortex as the ground truth for the activated brain areas in the SSVEP experiment from an independent structural MRI and fMRI studies, FIG. 9 shows ROC curves used to quantify the sensitivity and specificity of detecting the estimated 15-Hz neuronal current oscillation in the visual cortex using either OBS or DMH methods in EEG source modeling. For EPI-EEG, the area under the ROC curve (AUC) value for OBS and DMH was 0.85 and 0.87, respectively. For SMS-EPI-EEG, the AUC value for OBS and DMH was 0.93 and 0.98, respectively. For Inside-MRI, the AUC value for OBS and DMH was 0.91 and 0.97, respectively. The AUC increased progressively from EPI-EEG, SMS-Inl-EEG, and Inside-MRI. In all three conditions, DMH consistently had a larger AUC value than OBS.

The present example implementations of the DMH method for modelling the ballistocardiogram (BCG) is based on recorded EKG data and the subtraction of the BCG artifact from EEG data recorded inside an MRI system. As noted above, the performance of the DMH method was systematically characterized in comparison to the conventional OBS method in EEG BCG artifact suppression for two types of EEG experiments. First, a steady-state visual evoked response experiment was undertaken to record EEG data inside an MRI system either concurrently with EPI, with fast MRI, or in the static magnetic field without performing MRI. The DMH approach was stable across choices of parameter combinations (FIG. 4). Compared to OBS, DMH significantly improved time courses (FIG. 5A) and their 15-Hz oscillations (FIG. 5B) from occipital electrodes over the scalp in BCG suppression. Source modeling of SSVEPs revealed a stronger transient and oscillatory neuronal current in the visual cortex with DMH than with OBS (FIGS. 4 and 5). The estimated 15-Hz oscillation in the visual cortex was found to be about 30% stronger with DMH than with OBS in SMS-Inl-EEG and Inside-MRI conditions, respectively (FIG. 8). An ROC analysis showed that use of DMH resulted in higher power in detecting the oscillatory visual cortex neuronal activity in all three EEG acquisition scenarios (FIG. 9). In summary, the proposed DMH method reduced BCG artifacts further than was possible with the OBS method in the experimental conditions that were assessed, resulting in EEG signal time courses that were more related to neuronal activity.

The DMH method likely outperformed OBS in BCG artifact suppression because DMH adaptively identified BCG artifacts by pooling EEG recordings with similar cardiac dynamics, while accounting for variability in cardiac phase and signal amplitudes within the pool. The DMH method assumes that time instants of similar EKG dynamics have similar EEG BCG artifacts. This assumption was supported by early studies on the origin of BCG artifacts induced by the cardiac-related motion of the head and electrodes in a static magnetic field. One limitation of DMH is that the method requires identification of EKG events correlated to EEG signals. If the neuronal responses of interest are asynchronous with cardiac cycles, then DMH is expected to reveal these responses by adaptively synthesizing BCG artifacts from the EEG data with similar cardiac dynamics. The other limitation of DMH is the need to record the EKG, which is used to identify cardiac phase in modeling the BCG artifact. The EKG recording is also required by OBS, but not by the harmonic regression method.

The performance difference between DMH and OBS became progressively smaller from Inside-MRI, SMS-Inl-EEG, and EPI-EEG. This is likely due to the residuals in GA suppression, which is the least and largest in Inside-MRI and EPI-EEG, respectively. A better performance by DMH than OBS was consistently observed in evoked responses. Therefore, it may be beneficial to employ DMH to process EEG data with MRI collected using a minimal MRI acquisition time and an acceptable spatiotemporal resolution and a field-of-view to obtain high-quality EEG and MRI at the same time.

As noted above, the present example implementation of the DMH method is computationally efficient: it took less than 10 s to complete the BCG suppression on 32-channel EEG data recorded at 5,000 Hz for approximately 8 minutes. The calculation was performed by a CPU without the need for dedicated parallel processors or large memory capacity. In comparison, OBS took about 70 s to complete BCG suppression for the same data set using the same computational resource. The approximately 7-fold higher computational load in OBS, as compared to DMH, is due to use of Principal Component Analysis in OBS. Higher computational demand is common across component analysis methods.

In the present example experimental study, OBS was taken as the benchmark to test DMH performance. Other alternatives for advanced BCG suppression include harmonic regression and deep learning strategies such as BCGnet. However, harmonic regression gave an evoked response similar to OBS processing for EEG recorded inside a 3T magnet in the absence of MRI, an ideal case to avoid potential residual errors that arise from GAs during EEG-MRI. Given concurrently recorded EPI and EEG data inside a 3T MRI system, the BCGnet approach and OBS generated similar evoked responses even though the response variability was smaller using BCGnet than OBS. In contrast, in the present example DMH implementation provided more specific evoked responses.

Like OBS, DMH depends on the availability of the EKG to estimate time instants in terms of phases of cardiac cycles. Therefore, the quality of EKG affects the performance of DMH. Component analysis methods, however, assume either statistical independence or uncorrelation (orthogonality) between signal and noise time series to separate neuronal activity from measurement artifacts. The DMH method does not rely on these assumptions, nor does it require substantial ancillary hardware to record signals specific to noise processes. A typical setup of MRI-compatible EEG with EKG has been found by the present inventor to suffice for the suppression of BCG noise and the recovery of neuronal signals.

The present example experimental study investigated EEG data corrupted by BCG inside a 3T MRI scanner. The BCG artifact has been shown to scale with MRI field strength, suggesting that EEG data recorded in 7T MRI systems may have higher quality after DMH processing than OBS processing.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

Therefore what is claimed is:

1. A system for performing suppression of ballistocardiogram artifacts in electroencephalography data (EEG data), the system comprising:
processing circuitry comprising at least one processor and associated memory, said memory comprising instructions executable by said at least one processor for performing operations comprising:
receiving the EEG data and corresponding electrocardiogram data (EKG data);
processing the EKG data to identify a plurality of cardiac cycles, each cardiac cycle having an associated EKG waveform segment;
generating a modeled value of the EEG data for a selected latency value within a selected cardiac cycle having an associated EKG waveform segment, the modeled value being associated with the ballistocardiogram artifact contribution to the EEG data, by:
processing EKG waveform segments of cardiac cycles other than the selected cardiac cycle to identify one or more similar cardiac cycles that have similar EKG waveform segment values, proximal to the selected latency value, to those of selected cardiac cycle; and
employing values of the EEG waveforms of the similar cardiac cycles at the selected latency value to generate a modeled value of the EEG waveform at the selected latency value for the selected cardiac cycle; and
subtracting the modeled value of the EEG waveform from the EEG data at the selected latency value within the selected cardiac cycle to obtain corrected EEG data having BCG artifact suppressed at the selected latency value.

2. The system according to claim 1 said processor is configured such that processing EKG waveform segments of cardiac cycles other than the selected cardiac cycle to identify one or more similar cardiac cycles that have similar EKG waveform segment values, proximal to the selected latency value, to those of selected cardiac cycle, comprises:
obtaining, for each cardiac cycle, a set of EKG values corresponding to a set of latency values proximal to the selected latency value;
processing the sets of EKG values of cardiac cycles other than the selected cardiac cycle to identify one or more similar cardiac cycles that have a set of EKG values similar to those of selected cardiac cycle.

3. The system according to claim 2 wherein said processor is configured such that the one or more similar cardiac cycles are identified according to a distance-based similarity measure associated with a multi-dimensional representation of each set of EKG values, with each EKG value of a given set of EKG values being associated with a different dimension.

4. The system according to claim 3 wherein said processor is configured such that the distance-based similarity measure is computed according to Euclidean distance.

5. The system according to claim 1 wherein said processor is configured such that contributions of the EEG waveform values corresponding to the similar cardiac cycles are weighted according to similarity of the EKG waveform values to those of the selected cardiac cycle at the selected latency value to generate modeled value of the EEG waveform .

6. The system according to claim 1 wherein said processor is configured such that the latency is determined relative to a QRS complex associated with each cardiac cycle.

7. The system according to claim 1 wherein said processor is further configured to perform BCG artifact suppression at a plurality of latency values within the selected cardiac cycle.

8. The system according to claim 1 wherein said processor is further configured to perform BCG artifact suppression at a plurality of latency values within a plurality of selected cardiac cycles.

9. The system according to claim 1 further comprising:
an EKG sensor; and
an EEG electrode array;
wherein said processing circuitry is operatively coupled to said EKG sensor and said EEG electrode array.

10. The system according to claim 9 further comprising a magnetic resonance imaging scanner, wherein said processing circuitry is operatively coupled to said magnetic resonance imaging scanner.

11. A system for performing suppression of ballistocardiogram artifacts in electroencephalography data (EEG data), the system comprising:
processing circuitry comprising at least one processor and associated memory, said memory comprising instructions executable by said at least one processor for performing operations comprising:
receiving the EEG data and corresponding electrocardiogram data (EKG data);
processing the EKG data to identify a plurality of cardiac cycles, each cardiac cycle having an associated EKG waveform segment;
employing a temporal mapping to map the EKG data to a mapped EKG dataset such that each EKG waveform segment of the mapped EKG dataset is characterized by a respective cardiac cycle index and a latency parameter denoting latency within the respective cardiac cycle;
processing the mapped EKG dataset to generate, for each cardiac cycle, a corresponding multi-dimensional manifold, each dimension of the manifold being associated with a respective mapped EKG dataset having a unique time lag;
processing the manifolds to identify one or more similar cardiac cycles that have similar values, at a selected latency value, to that of a selected cardiac cycle at the selected latency value;
employing values of the EEG waveforms of the similar cardiac cycles at the selected latency value to generate a modeled value of the EEG waveform at the selected latency value for the selected cardiac cycle; and
subtracting the modeled value of the EEG waveform from the EEG data at the selected latency value within the selected cardiac cycle to obtain corrected EEG data having BCG artifact suppressed at the selected latency value.
